(19) United States
(12) Patent Application Publication
(10) Pub. No.: US 2011/0137219 A1
(43) Pub. Date: Jun. 9, 2011

(54) Method and apparatus to grow hair

(76) Inventor: William An, Flushing, NY (US)

(21) Appl. No.: 12/632,576

(22) Filed: Dec. 7, 2009

(51) Int. Cl. A61H 7/00; A61B 17/00

(52) U.S. Cl. 601/134; 128/898; 601/1

(57) ABSTRACT

A method for growing hair includes the steps of detecting a trigger point in proximity to the area for hair growth, reducing the trigger point in proximity to the area for hair growth, and massaging the area for hair growth. The step of massaging may include manual massaging and may include electronically massaging.

### FIELD OF THE INVENTION

The present invention relates to hair growth, and more particularly to a method and apparatus for promoting regeneration of hair growth.

### BACKGROUND

Alopecia (i.e., balding and thinning of hair, especially on the scalp) is common among many people for a variety of known (e.g., heredity) and unknown and/or unrecognized reasons. For their appearance and/or a variety of other reasons, some people who are balding or have thinning hair on all or parts of their scalp would rather have hair on those areas. As a result, a variety of treatments have been tried to stop the loss of hair, and preferably to regenerate new hair growth where hair has been lost.

For example, Upjohn Company of Kalamazoo, Mich., USA, has obtained wide success with the topical dermatological for treatment of alopecia which it sells under its ROGAINE™ trademark. However, even as successful as that treatment has been, there are still many, many people on whom the treatment does not work or does not work satisfactorily (e.g., the texture of the regenerated hair may be unsatisfactory), or to whom the treatment is too expensive.

In short, the reality is that despite the long felt need for adequate treatment, there is still no adequate treatment form many people who have, but do not want to have, balding or thinning hair.

In the medical field of muscular and myofacial pain, Dr. Janet Travell is recognized as the leading pioneer. She began her work in the 1940s, in the United States. She developed an entire branch of medicine, known as “Myofacial Therapy”. She was President John F. Kennedy's personal physician, and successfully treated his chronic back condition. Myofacial trigger point therapy is based almost entirely on Dr. Travell's work, and is the most effective modality used today by clinicians and therapists who treat muscular and myofacial dysfunction and pain. It is a therapeutic technique that involves the systematic application of pressure to tender muscles and myofacial tissue in order to relieve pain and dysfunction.

Myofacial tissue (also called “fascia”) wraps around muscle tissue, muscle fibers, bundles of fibers, and the muscles themselves. It then continues on to form tendons and ligaments. Thus, fascia and muscle tissues are interwoven. When muscles and fascia are in their normal, relaxed state, the layers of facial tissue, muscle fibers, bundles of fibers, and the muscles themselves all glide alongside one another. When muscular dysfunction is present, muscles contract (cramp) to varying degrees, which is known as “going into spasm”. Moreover, facial dysfunction always arises from muscular dysfunction.

Ordinary muscle cramps and contractions release with movement, that is, stretching of the affected musculature. When the muscle is locked into a deep and painful spasm, it forms a “trigger point.” (Another term commonly used instead of trigger point is “contraction knot.”) Trigger points do not release with movement (stretching). Instead, they are locked into a strong state of contraction. This contraction is essentially a localized hardening of the muscle tissue and associated fascia.

In the medical field of muscular and myofacial pain, Dr. Janet Travell is recognized as the leading pioneer. She began her work in the 1940s, in the United States. She developed an entire branch of medicine, known as “Myofacial Therapy”. She was President John F. Kennedy's personal physician, and successfully treated his chronic back condition. Myofacial trigger point therapy is based almost entirely on Dr. Travell's work, and is the most effective modality used today by clinicians and therapists who treat muscular and myofacial dysfunction and pain. It is a therapeutic technique that involves the systematic application of pressure to tender muscles and myofacial tissue in order to relieve pain and dysfunction.

Myofacial tissue (also called “fascia”) wraps around muscle tissue, muscle fibers, bundles of fibers, and the muscles themselves. It then continues on to form tendons and ligaments. Thus, fascia and muscle tissues are interwoven. When muscles and fascia are in their normal, relaxed state, the layers of facial tissue, muscle fibers, bundles of fibers, and the muscles themselves all glide alongside one another. When muscular dysfunction is present, muscles contract (cramp) to varying degrees, which is known as “going into spasm”. Moreover, facial dysfunction always arises from muscular dysfunction.

Ordinary muscle cramps and contractions release with movement, that is, stretching of the affected musculature. When the muscle is locked into a deep and painful spasm, it forms a “trigger point.” (Another term commonly used instead of trigger point is “contraction knot.”) Trigger points do not release with movement (stretching). Instead, they are locked into a strong state of contraction. This contraction is essentially a localized hardening of the muscle tissue and associated fascia.

Trigger point therapy is well known and widely used in the medical field on the muscles of the body, for example, the back or shoulder.

The present invention is directed toward overcoming one or more of the problems set forth above.

### SUMMARY

A method for growing hair includes the steps of detecting a trigger point in proximity to the area for hair growth, reducing the trigger point in proximity to the area for hair growth, and massaging the area for hair growth.

The step of massaging may include manual massaging and may include electronically massaging.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates the steps of the present invention.

### DETAILED DESCRIPTION

Trigger points or trigger sites are described as hyperirritable spots in skeletal muscle that are associated with palpable nodules in taut bands of muscle fibers. Trigger point researchers believe that palpable nodules are small contraction knots and a common cause of pain. Compression of a trigger point may elicit local tenderness, referred pain, or local twitch response. The local twitch response is not the same as a muscle spasm. This is because a muscle spasm refers to the entire muscle entirely contracting whereas the local twitch response also refers to the entire muscle but only involves a small twitch, no contraction.

The trigger point model states that unexplained pain frequently radiates from these points of local tenderness to broader areas, sometimes distant from the trigger point itself. Practitioners claim to have identified reliable referred pain patterns, allowing practitioners to associate pain in one location with trigger points elsewhere. Many chiropractors and massage therapists find the model useful in practice, but the medical community at large has not embraced trigger point therapy. Although trigger points do appear to be an observable phenomenon with defined properties, there is a lack of a consistent methodology for diagnosing trigger points^{[2]} and a dearth of theory explaining how trigger points arise and why they produce specific referred pain patterns.

The term “trigger point” was coined in 1942 by Dr. Janet Travell to describe a clinical finding with the following characteristics:
Pain related to a discrete, irritable point in skeletal muscle or fascia, not caused by acute local trauma, inflammation, degeneration, neoplasm or infection.
The painful point can be felt as a tumor or band in the muscle, and a twitch response can be elicited on stimulation of the trigger point.
Palpation of the trigger point reproduces the patient's complaint of pain, and the pain radiates in a distribution typical of the specific muscle harboring the trigger point.
The pain cannot be explained by findings on neurological examination.
Trigger points have a number of qualities. They may be classified as active/latent and also as key/satellites and primary/secondary.
An active trigger point is one that actively refers pain either locally or to another location (most trigger points refer pain elsewhere in the body along nerve pathways). A latent trigger point is one that exists, but does not yet refer pain actively, but may do so when pressure or strain is applied to the myoskeletal structure containing the trigger point. Latent trigger points can influence muscle activation patterns, which can result in poorer muscle coordination and balance.
A key trigger point is one that has a pain referral pattern along a nerve pathway that activates a latent trigger point on the pathway, or creates it. A satellite trigger point is one which is activated by a key trigger point. Successfully treating the key trigger point often will resolve the satellite and return it from being active to latent, or completely treating it too.
In contrast, a primary trigger point in many cases will biomechanically activate a secondary trigger point in another structure. Treating the primary trigger point does not treat the secondary trigger point.
Activation of trigger points may be caused by a number of factors, including acute or chronic muscle overload, activation by other trigger points (key/satellite, primary/secondary), disease, psychological distress (via systemic inflammation), homeostatic imbalances, direct trauma to the region, radiculopathy, infections and health choices such as smoking.
Trigger points can appear in many myofascial structures including muscles, tendons, ligaments, skin, joint capsule, periosteal, and scar tissue. When present in muscles there is often pain and weakness in the associated structures. These pain patterns in muscles follow specific nerve pathways and have been readily mapped to allow for identification of the causative pain factor. Many trigger points have pain patterns that overlap, and some create reciprocal cyclic relationships that need to be treated extensively to remove them.

Usually there is a taut band in muscles containing trigger points, and a hard nodule can be felt. Often a twitch response can be felt in the muscle by running your finger perpendicular to the muscle's direction; this twitch response often activates the “all or nothing” response in a muscle that causes it to contract. Pressing on an affected muscle can often refer pain. Clusters of trigger points are not uncommon in some of the larger muscles, such as the gluteus group (gluteus maximus, gluteus medius, and gluteus minimus). Often there is a heat differential in the local area of a trigger point, and many practitioners can sense that.

Treatment of trigger points may be by manual, Myofascial Trigger Point Therapy by a trained Myofascial Trigger Point Therapist, Myotherapy (deep pressure, massage or tapotement), mechanical vibration, pulsed ultrasound, electrostimulation, ischemic compression, injection (see below), dry-needling, “spray-and-stretch” using a cooling (vapocoolant) spray, Low Level Laser Therapy and stretching techniques that invoke reciprocal inhibition within the musculoskeletal system. Use of elbows, feet or various tools to direct pressure directly upon the trigger point often occurs, to save practitioner's hands.

A successful treatment protocol relies on identifying trigger points, resolving them and, if all trigger points have been deactivated, elongating the structures affected along their natural range of motion and length. In the case of muscles, which is where most treatment occurs, this involves stretching the muscle using combinations of passive, active, active isolated (AIS), muscle energy techniques (MET), and proprioceptive neuromuscular facilitation (PNF) stretching to be effective. Fascia surrounding muscles should also be treated, possibly with myofascial release, to elongate and resolve strain patterns, otherwise muscles will simply be returned to positions where trigger points are likely to re-develop.

The results of manual therapy are related to the skill level of the therapist. If trigger points are pressed too short a time, they may activate or remain active; if pressed too long or hard, they may be irritated or the muscle may be bruised, resulting in pain in the area treated. This bruising may last for a 1-3 days after treatment, and may feel like, but is not similar to, delayed onset muscle soreness (DOMS), the pain felt days after overexerting muscles. Pain is also common after a massage if the practitioner uses pressure on unnoticed latent or active trigger points, or is not skilled in myofascial trigger point therapy. Lack of reduction of pain after one to three treatment sessions by a trigger point practitioner should be referred to a medical professional. Evidence based medicine researchers have concluded evidence for the usefulness of trigger points in the diagnosis of fibromyalgia is thin. Fibromyalgia patients generally have multiple, recurring trigger points, typically in a quadrant or more of the body.

### Injection

Injections provide more immediate relief and can be effective when other methods fail.

Various injections can be used including saline, local anesthetics such as procaine hydrochloride (Novocain), steroids, and botulinum toxin. Injection with a low concentration, short acting local anesthetic (Procaine 0.5%) without steroids or adrenalin is recommended. High concentrations or long acting local anesthetics as well as epinephrine cause muscle necrosis. Use of steroids can cause tissue damage. Dry needling can be just as effective but causes more post-injection soreness. Botox is rarely indicated.

Despite the concerns about long acting agents, a mixture of lidocaine and marcaine is often used. A mixture of 1 part 2% lidocaine with 3 parts 0.5% bupivacaine (trade name: Marcaine) provides 0.5% lidocaine and 0.375% bupivacaine. This has the advantages of immediate anesthesia with lidocaine during injection to minimize injection pain while providing a longer duration of action with a lowered concentration of bupivacaine.

Sarapin can be used for trigger point injection. Blue Cross, Medica, HealthPartners and other health insurance companies in the US began covering Trigger Point injections in 2005.

### Self-Treatment

There are a number of ways to self-treat trigger points and these methods are described in numerous texts. Underlying any attempts at self-treatment should be a working knowledge of the area to be treated, especially with regard to the musculature, nerves, glands and vessels.

Trigger points in the male or female pelvis, such as found in chronic pelvic pain syndrome (CPPS), should be treated by physicians trained in the use of intra-rectal trigger point and myofascial release techniques.

### Risks

Treatment, whether by self or by a professional, has some inherent dangers. It may lead to damage of soft tissue and other organs. The trigger points in the upper quadratus lumborum, for instance, are very close to the kidneys and poorly administered treatment (particularly injections) may lead to kidney damage. Likewise, treating the masseter muscle may damage the salivary glands superficial to this muscle. Furthermore, some experts believe trigger points may develop as a protective measure against unstable joints.

U.S. Pat. No. 6,911,013, incorporated by reference in its entirety, discloses a method and apparatus of regenerating hair growth, including the steps of engaging a scalp area with an edge of a flexible member, reciprocating a portion of the flexible member through a stroke length along an axis of at least ½ inch at a rate of at least 1,500 strokes per minute, with the flexible member portion being spaced from the scalp area and the axis being oriented in a direction parallel to tangential to the engaged scalp area, and repeating the engaging and reciprocating steps until hair regeneration begins. An apparatus usable in such a method includes a handle, a drive secured to the handle and reciprocating along an axis relative to the handle, and a flexible massage member secured to the reciprocating drive. The massage member has an axial portion aligned with the axis, and a silicone massage portion extending laterally from the axial portion to a scalp engaging surface, with the massage portion being substantially flexible in the axial direction. The flexible massage member extends a lateral distance X and an axial distance Y, where X>Y, with the axial distance tapering down from a maximum axial distance at the axial portion to a minimum axial distance at the scalp engaging surface.

The device described by the above mentioned patent can be used to massage the area where hair rejuvenation is required. Alternatively, the massage can be performed by the user or another through finger pressure. The result of detecting the trigger point, eliminating or reducing the trigger point followed by massage either manually or by the use of the above device, is the regrowth or rejuvenation of hair.

The elimination of the trigger points allows the hair to regrow, and the massage of the air particles enhances their growth. The described procedure may be applicable to any part of the body, especially the scalp.

The above procedure eliminates the hotspots or areas of tissue where the temperature has been elevated due to the trigger points.

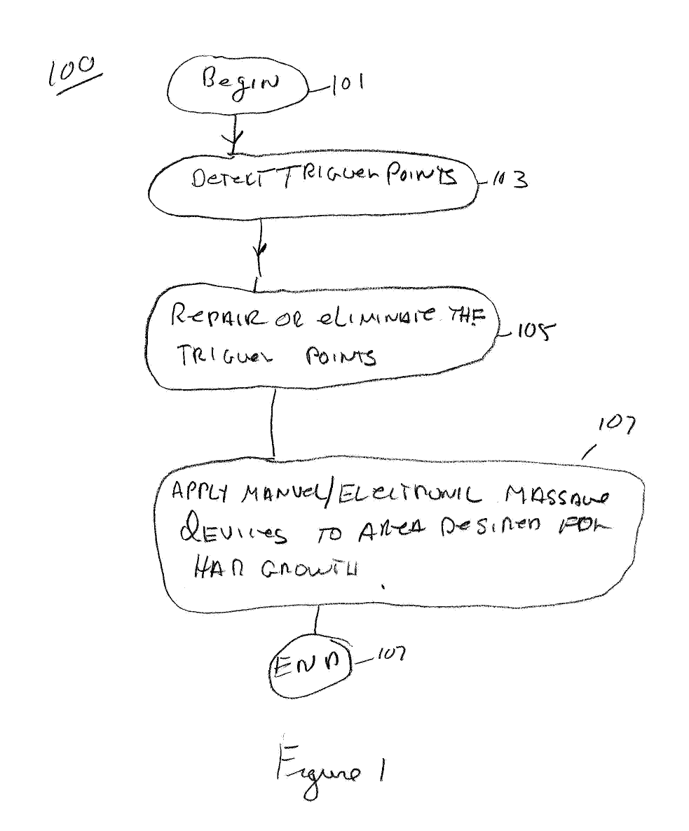

FIG. 1 illustrates a flow chart 100 of the steps of the invention, the flow chart 100 begins at step 101. From step 101, trigger points are detected in step 103. In step 105, the trigger points or trigger point is eliminated or reduced in order to reduce the hotspot or warm spot which may be inhibiting hair growth. In step 107, the area that hair growth is desired is massage either by an electronic device or manually. In step 109, the process ends and hair growth resumes or begins.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A method for growing hair, comprising the steps of:
detecting a trigger point in proximity to the area for hair growth;
reducing the trigger point in proximity to the area for hair growth;
massaging the area for hair growth.

2) A method for growing hair as in claim 1, wherein the step of massaging includes the step of manual massaging.

3) A method for growing hair as in claim 1, wherein the step of massaging includes the step of electronically massaging.
